(19) United States
(12) Patent Application Publication
(10) Pub. No.: US 2023/0065570 A1
(43) Pub. Date: Mar. 2, 2023

(54) Simulation Model Sample for Evaluation of Heat Treatment, and Method for Evaluating Heat Treatment Using Simulation Model Sample

(71) Applicant: HISAKA WORKS, LTD., Osaka-shi (JP)

(72) Inventor: Kota Ukai, Higashi-Osaka-shi (JP)

(21) Appl. No.: 18/049,383

(22) Filed: Oct. 25, 2022

Related U.S. Application Data
(62) Division of application No. 16/634,559, filed on Jan. 27, 2020.

(30) Foreign Application Priority Data
Jul. 26, 2017 (JP) 2017-144789

(51) Int. Cl. G01N 25/48; G01N 30/00

(52) U.S. Cl. CPC G01N 25/48; G01N 30/00; A23L 3/10

(57) ABSTRACT

Provided is a simulation model sample for evaluation of heat treatment including a porous water absorbing material that is flexible and deformable; and a container that is configured to be able to contain the porous water absorbing material having water absorbed therein. Also provided is a method for evaluating heat treatment using a simulation model sample including a step of allowing a flexible and deformable porous water absorbing material to absorb water, and the porous water absorbing material to be contained in a container, to produce a simulation model sample; and a step of subjecting the simulation model sample to heat treatment, while measuring a temperature inside the simulation model sample.

### CROSS-REFERENCE TO RELATED APPLICATIONS

This application is a divisional of U.S. patent application Ser. No. 16/634,559, filed Jan. 27, 2020, which is the United States national phase of International Application No. PCT/JP2018/003826 filed Feb. 5, 2018, and claims priority to Japanese Patent Application No. 2017-144789, the disclosures of which are hereby incorporated by reference in their entirety.

### FIELD

The present invention relates to a simulation model sample for evaluation of heat treatment and a method for evaluating heat treatment using a simulation model sample.

### BACKGROUND

In manufacturing packed food, such as canned food and retort food, the manufactured packed food is heat sterilized. Whether or not a certain heating condition is suitable for food sterilization is generally evaluated using an F-value, which is a sterilization value expressed in a relation between temperature and time. That is, in the case where the temperature history of food under a certain heating condition meets a predetermined F-value, it can be evaluated that the heating condition is suitable for food sterilization. For example, an F-value of retort food is required to be equivalent to 4 minutes at 120.0° C. or greater, pursuant to the Food Sanitation Act.

Meanwhile, when food is subjected to heat treatment, nutritional substances such as proteins and vitamins present in food decompose; thus, the heating conditions need to be set in consideration of influences on such active substances. The same applies to heating of an object other than food. For example, heat treatment of a medicinal product or a medical device may have an adverse effect on, for example, the pharmaceutical substances of the medicinal product or on the materials forming the medical device. It is thus required to evaluate the heating conditions of an object to be processed also from other viewpoints than sterilization.

As a method for evaluating the heating temperature of an object to be heated, a simulation method for calculating the heating temperature of the object to be processed using a computer has been conventionally known (see, for example, Patent Literature 1).

### CITATION LIST

### Patent Literature

Patent Literature 1: JP 3071412 B

### SUMMARY

### Technical Problem

However, the calculation result obtained by the simulation cannot completely reproduce the temperature of the object to be processed which has been actually subjected to heat treatment. Therefore, in order to verify the accuracy of the calculation result of the simulation, it is necessary to measure the temperature of the object to be processed by actually subjecting the object to heat treatment, and compare the calculation result obtained by the simulation with the result obtained by the actual measurement to verify the accuracy, and in some cases, correct various setting conditions in the simulation.

An object of the present invention is to provide a simulation model sample for evaluation of heat treatment that is suitably usable, as a substitute for an actual product that is an object to be processed, in the case where the temperature of the object to be processed is measured by actually heating the same, for the purpose of, for example, verifying the accuracy of the simulation of the heat treatment of the object to be processed, as mentioned above. Another object of the present invention is to provide a method for evaluating heat treatment using a simulation model sample to evaluate the thermal effect of heat treatment on an object to be processed.

### Solution to Problem

A simulation model sample for evaluation of heat treatment according to the present invention includes: a porous water absorbing material that is flexible and deformable; and a container that is configured to be able to contain the porous water absorbing material having water absorbed therein.

Further, a method for evaluating heat treatment using a simulation model sample according to the present invention includes: a step of allowing a flexible and deformable porous water absorbing material to absorb water, and the porous water absorbing material to be contained in a container, to produce a simulation model sample; and a step of subjecting the simulation model sample to heat treatment, while measuring a temperature inside the simulation model sample.

Further, a method for evaluating heat treatment using a simulation model sample according to the present invention includes: a step of allowing a flexible and deformable porous water absorbing material to absorb water and a substance whose properties vary due to heating, and the porous water absorbing material to be contained in a container, to produce a simulation model sample; a step of subjecting the simulation model sample to heat treatment; and a step of measuring a variation of properties of the substance before and after the heat treatment.

Further, a method for evaluating heat treatment using a simulation model sample according to the present invention includes: a step of producing a simulation model sample including a flexible and deformable porous water absorbing material, a small container containing a substance whose properties vary due to heating, the small container being embedded in the porous water absorbing material having water absorbed therein, and a container in which the porous water absorbing material is contained; a step of subjecting the simulation model sample to heat treatment; and a step of measuring a variation of properties of the substance before and after the heat treatment.

As one form of the present invention, a sponge may be employed as the porous water absorbing material. Since various types of sponges having different water absorbing properties are easily available, and a sponge having a void ratio and an amount of water absorption varying according to the type thereof can be used, the sponge is advantageous in that it can easily reproduce the physical properties similar to the actual product.

As one form of the present invention, the simulation model sample may include a substance whose properties vary due to the effect of heat. When the porous water absorbing material having the substance absorbed therein is subjected to heat treatment, it is possible to perform more specific evaluation of the temperature inside the simulation model sample by measuring a variation in the amount or ratio of the substance before and after the heat treatment.

Advantageous Effect of Invention

According to the present invention, it is possible to provide a simulation model sample for evaluation of heat treatment that is suitably usable as a substitute for an actual product that is an object to be processed, in the case where the temperature of the object to be processed is measured by actually heating the same, for the purpose of, for example, verifying the accuracy of the simulation for the evaluation of the heat treatment of food, medicinal product, or the like, as aforementioned above. Further, according to the present invention, it is possible to provide a method for evaluating heat treatment using a simulation model sample can be provided.

### BRIEF DESCRIPTION OF THE DRAWINGS

FIG. 1 is a perspective view of a simulation model sample for evaluation of heat treatment of an embodiment according to the present invention.

FIG. 2 is a cross-sectional view taken along the line II-II in FIG. 1.

FIG. 3 is a cross-sectional view taken along the line III-III in FIG. 1.

### DESCRIPTION OF EMBODIMENTS

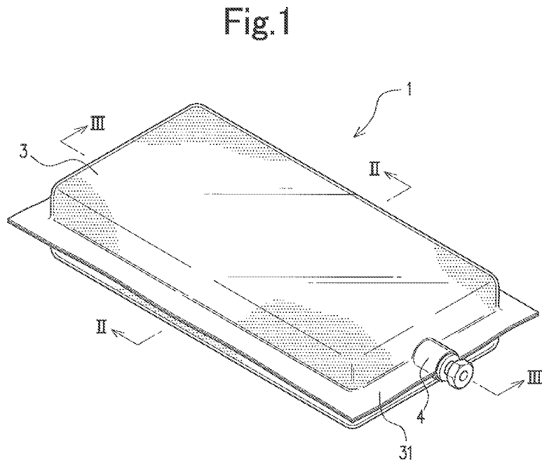

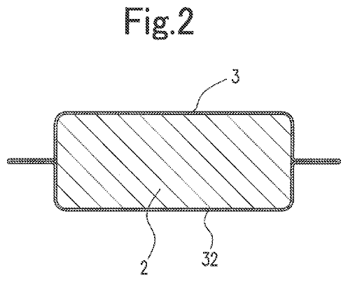

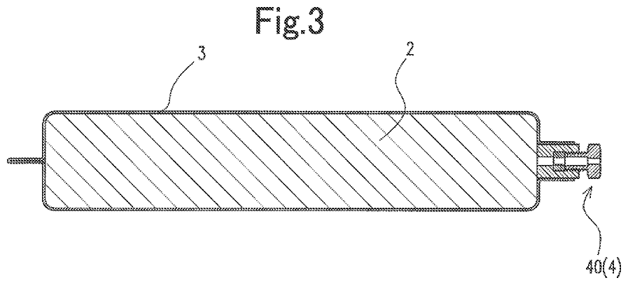

Hereinafter, an embodiment of the present invention will be described with reference to the drawings. A simulation model sample 1 for evaluating heat treatment of this embodiment is a simulation model sample, configured to be a so-called retort pouch food as an actual product, and, as shown in FIG. 1 to FIG. 3, includes a porous water absorbing material 2 that is flexible and deformable and a container 3 that contains the porous water absorbing material 2 having water absorbed therein and is sealed.

As the flexible and deformable porous water absorbing material 2, a flexible material capable of absorbing water and retaining it can be used. In terms of the intended use in heat treatment for the purpose of sterilization of food, medicinal product, or the like, it is preferable that the porous water absorbing material have a heat-resistant temperature of about 120° C. and itself not decompose, or its properties do not vary even when the porous water absorbing material is heated to such a temperature. Specifically, as the porous water absorbing material, sponge, nonwoven fabric, towel fabric, or the like can be suitably used. Examples of the sponge include synthetic resin sponge such as polyurethane sponge, nylon sponge, and melamine sponge, and natural material sponge such as cellulose sponge. Examples of the nonwoven fabric include nonwoven fabric composed of chemical fibers such as nylon and polyester, and nonwoven fabric composed of natural fibers such as cotton, wool, and hemp.

The porous water absorbing material 2 can be used with its shape and size desirably adjusted, and is preferably made to have the same shape and the same size as those of the actual product. When the size of the actual product is large, the porous water absorbing material 2 composed of a plurality of the same type of materials stacked together may be used.

Alternatively, two or more types of the aforementioned materials can be combined to provide a porous water absorbing material with properties partially different from each of those original materials. More specifically, a simulation model sample can be composed of a first porous water absorbing material as an inner region including a central portion of the simulation model sample and a second porous water absorbing material as an outer region surrounding the inner region. The simulation model sample having such a structure can more accurately reproduce the temperature even if the actual product has physical properties different in the inner region and the outer region.

As the container 3, a container capable of containing the porous water absorbing material 2 that is adjusted to have a desired shape and is kept in a state of absorbing water can be used, and a container capable of sealing its inside can be preferably used. In order to allow the simulation model sample 1 to have a temperature close to the temperature of the actual product to be heated when the simulation model sample 1 has been subjected to heat treatment, it is preferable that the container be the same as or similar to the container of the actual product to be heated. For example, when the simulation model sample is configured to correspond to retort food, a container in the form of film used as a container for the retort food is preferable, and when the simulation model sample is configured to correspond to canned food, a container made of metal is preferable. Also in the case where a medicinal product, quasi-medicinal product, or the like is subjected to heat treatment, a container that is the same as or similar to the container to contain the medicinal product can be preferably used.

The container 3 according to this embodiment is made of a film material and is configured to allow a sponge as the porous water absorbing material 2 having water absorbed therein to be contained inside the container, and an opening 31 of the container 3 is thermally fused and closed. Thereby, the container 3 contains the porous water absorbing material 2 and is sealed.

Further, an insertion port 4 for inserting a thermometer into the inside of the container 3 is provided in the simulation model sample 1 according to this embodiment. Specifically, the insertion port 4 is configured with an insertion valve 40 having an openable and closable communication hole fixed by being sandwiched between the thermally fused film materials at the opening 31 of the container 3.

The insertion port 4 provided in the simulation model sample 1 allows the thermometer to be inserted into the inside of the simulation model sample 1 through itself, and thus enables easy measurement of the temperature inside of the container when the simulation model sample 1 is subjected to heat treatment.

A substance whose properties vary due to heating may be added in the porous water absorbing material 2. Example of such a substance may include, for example, vitamins, proteins, enzymes, bacteria, fungi, nutrient cells, and thermosensitive polymer materials. In this embodiment, only one of the substances or two or more of the substances can be used in combination. Here, the expression “the substance whose properties vary” means that the physical properties of the substance vary, or that the substance decomposes and disappears.

Examples of the vitamins include vitamin A, vitamin B, vitamin C, vitamin D, vitamin E, vitamin K, and riboflavin lactate. Examples of vitamin B include vitamin B1 derivatives such as thiamine and its salt, and vitamin B complexes containing vitamin B2, vitamin B6, vitamin B12, biotin, pantothenic acid, nicotinic acid, folic acid, etc.

Particularly, it is preferable to use a substance whose correlation of its variation in properties, that is, decay or generation, relative to the heating, that is, the temperature and the time has been already known. When this substance is used when comparison is made between the simulation result and the analysis result of the measured value, it is possible to easily achieve accurate evaluation.

Next, an embodiment of the method for evaluating heat treatment according to the present invention will be described. In the first step of this embodiment, a sponge as a flexible porous water absorbing material is adjusted to have a size and a shape substantially the same as those of the actual product to be heated. When the product size is large, a plurality of sponges of the same type may be stacked together to be used. Alternatively, a plurality of types of sponges may be combined to be used. Then, the sponge is impregnated with water, contained in the container, and the opening of the container is sealed by a vacuum packaging machine to prepare a simulation model sample. When the opening of the container is sealed, an insertion valve for inserting a thermometer as aforementioned is preferably mounted by being held between the film materials of the fused portion.

In the second step, the thermometer is mounted on the prepared simulation model sample and placed in a heat sterilization equipment. When an insertion valve is provided, the thermometer is inserted into the container from the insertion valve and fixed so as to enable the measurement of the temperature at a desired position such as a center or an outer region of the simulation model sample. When no insertion valve is provided, it may be configured that the thermometer is inserted through a hole pierced in a part of the container, and the thermometer is installed therethrough so as to enable the measurement of the temperature of a desired region.

Then, the simulation model sample is subjected to the heat treatment in accordance with a predetermined heating condition, followed by measuring the temperature inside the simulation model sample by the thermometer installed inside the simulation model sample.

The measured result of temperature of the simulation model sample thus obtained is compared with the measured result of temperature of the actual product to be heated, and if the difference therebetween is large, a new simulation model sample may be prepared by changing the type of sponge. For example, when the temperature difference between the center and the surrounding region of the actual product is larger than that in the simulation model sample, it would be necessary to change the sponge to a sponge having a larger porosity and a smaller thermal conductivity. To the contrary, when the temperature difference between the center and the surrounding region of the actual product is smaller than that in the simulation model sample, it would be necessary to change the sponge a sponge having a smaller porosity and a larger thermal conductivity.

Since the measured result of temperature of the simulation model sample thus obtained is much closer to the measured result of temperature of the actual product to be heated, it can be used as a comparison target to verify the calculation result obtained by the simulation, for example. The simulation is not particularly limited and may be any conventionally known simulation. For the calculation of the substance whose properties vary due to heating, it is possible to use the same method as, for example, a method for calculating an F-value known in the sterilization of food.

In the simulation model sample of this embodiment, a flexible porous water absorbing material is contained in the container so that the convection of water in the container during the heat treatment is suppressed. Therefore, the convection heat transfer is less likely to occur inside the container, and the temperature gradient due to the conduction heat transfer is likely to be formed, compared with the case where a simulation model sample containing only water without using a porous water absorbing material is used. This is a similar state occurring in the product containing solid or highly viscous contents such as retort food or canned food, and therefore produces an effect of easily reproducing a temperature distribution similar to the actual product.

Further, since the simulation model sample used in this embodiment contains the flexible porous water absorbing material in the container, it produces an effect of easily installing, for example, a thermometer having a needle shaped tip or a sharp tip or various types of detector at an arbitrary position in the simulation model sample 1.

Now, another embodiment of the method for evaluating heat treatment of the present invention will be described. In the first step of this embodiment, a sponge as the flexible porous water absorbing material is adjusted to have a size and a shape substantially the same as those of the actual product to be heated. When the product size is large, a plurality of sponges may be stacked together to be used, or a plurality of types of sponges may be combined together to be used. Then, the sponge is impregnated with a mixed liquid of water and a substance whose properties vary due to heating, and contained in a container, followed by sealing the opening of the container with a vacuum packaging machine, to thereby produce a simulation model sample.

In the succeeding second step, the heat treatment of the simulation model sample is performed in accordance with a predetermined heating condition in the same manner as the aforementioned first embodiment.

In the third step, the mixed liquid absorbed in the porous water absorbing material is recovered from the simulation model sample after the heat treatment, followed by measuring a variation of properties of the substance. Thereby, a variation of the properties of the substance before and after the heat treatment, for example, an amount of the substance reduced by heating, can be measured.

Although the effect of heat treatment on the actual product may be evaluated by using the actual product, it is not easy to measure only the variation of properties of a certain substance, which vary due to heating, because various substances are included in the actual product as contents. On the other hand, the simulation model sample of this embodiment includes only the substance whose properties vary due to heating, so that a variation of the properties of the substance before and after the heat treatment can be easily measured, and the effect of heat treatment on the product can be more accurately evaluated.

Further, as a means for suppressing the convection of the liquid in the container, a method of adding a thickener to water without using a flexible porous water absorbing material may be employed. However, when the thickener and the substance whose properties vary due to heating are mixed, it becomes difficult to separate the substance from the thickner after the heat treatment, and the reduced amount of the substance cannot be accurately measured. On the other hand, since the flexible porous water absorbing material of the simulation model sample of this embodiment is used after it is impregnated with a mixed liquid of water and the substance whose properties vary by heating; thus, the mixed liquid of water and the substance can be easily separated from the porous water absorbing material by, for example, squeezing the porous water absorbing material after the heat treatment.

The amount of the substance whose properties vary due to heating may be determined by measuring the total amount before and after the heat treatment, or by measuring a part thereof, i.e., the amount (concentration) of the substance per unit of liquid.

Also in this embodiment, an insertion valve may be mounted, when the opening of the container is sealed, to allow a thermometer to be mounted via the insertion valve. The thermometer placed inside the simulation model sample enables measurement of the temperature inside the simulation model sample, and thereby enables evaluation by the temperature measurement in addition to the evaluation by the amount of the substance whose properties vary by heating.

Now, still another embodiment of the heat treatment evaluation method according to the present invention will be described. In the first step of this embodiment, a sponge as the flexible porous water absorbing material is adjusted to have the same size and the same shape as those of the actual product to be heated. When the product size is large, a plurality of sponges may be stacked together to be used, or a plurality of types of sponges may be combined together to be used. Meanwhile, a substance whose properties vary due to heating is contained in a small container that is relatively small compared to the porous water absorbing material, and the small container is embedded in the porous water absorbing material. Then, the porous water absorbing material with water absorbed therein is contained in a container, followed by sealing the opening of the container with a vacuum packaging machine to thereby produce a simulation model sample.

The substance whose properties vary due to heating can be contained in a small container together with a solvent such as water. The small container is not particularly limited as long as it is relatively small compared to the porous water absorbing material and the substance whose properties vary due to heating can be sealed therein and taken out therefrom. As the small container, for example, a capsule-type container with a capacity of about several ml can be suitably employed. Further, a plurality of small containers can be used at the same time and can be embedded in different parts of the porous water absorbing material.

In the second step, the heat treatment of the simulation model sample is performed in accordance with a predetermined heating condition in the same manner as the other embodiments.

In the third step, the amount of the substance whose properties vary due to heating is measured by taking out the small container embedded in the porous water absorbing material from the simulation model sample subjected to heat treatment and recovering the substance therefrom.

In this embodiment, the small container containing the substance whose properties vary due to heating can be embedded in a desired part of the porous water absorbing material; thus, a variation of the properties of the substance before and after the heat treatment can be measured for each part. That is, it becomes possible to individually measure and evaluate the effect of heat for each part of the simulation model sample, for example, for the center part and the periphery part.

The simulation model sample and the method for evaluating heat treatment described in the aforementioned embodiments are only examples of the present invention, and the present invention is not limited to the aforementioned embodiments. The aforementioned embodiments are described by taking, for example, the case where the container 3 is made of a film material, but the container of the present invention is not limited to a film material, and for example, a container made of plastic or metal can be employed.

Further, in the aforementioned embodiments, the inside of the container is sealed under reduced pressure by using a vacuum packaging machine, but the container intentionally held in an aerated state with air remaining in the container may be sealed by a common heat sealer, without reducing the pressure inside the container. Further, the porous water absorbing material of the aforementioned embodiment is impregnated with the mixed liquid of water and a substance whose properties vary due to heating, but may be impregnated with the substance separately from water.

As aforementioned, the simulation model sample for evaluation of heat treatment according to the present invention includes: a porous water absorbing material that is flexible and deformable; and a container that is configured to contain the porous water absorbing material having water absorbed therein.

Further, the method for evaluating heat treatment using a simulation model sample according to the present invention includes: a step of allowing a flexible and deformable porous water absorbing material to absorb water, and the porous water absorbing material to be contained in a container, to produce a simulation model sample; and a step of subjecting the simulation model sample to heat treatment, while measuring a temperature inside the simulation model sample.

Further, the method for evaluating heat treatment using a simulation model sample according to the present invention includes: a step of allowing a flexible and deformable porous water absorbing material to absorb water and a substance whose properties vary due to heating, and the porous water absorbing material to be contained in a container, to produce a simulation model sample; a step of subjecting the simulation model sample to heat treatment; and a step of measuring a variation of properties of the substance before and after the heat treatment.

According to the aforementioned method for evaluating heat treatment, convection of water absorbed in the flexible and deformable porous water absorbing material is suppressed even when the simulation model sample is heated; thus, a temperature gradient similar to that of the actual product to be heated, that is, food such as retort food or canned food, a jelly or creamy medicinal product or a quasi-medicinal product, or the like, is likely to be formed inside the simulation model sample. Therefore, the temperature approximate to the temperature of the actual product can be determined by measuring the temperature of the simulation model sample which has been subjected to the heat treatment. Accordingly, the present method is suitably used, for example, in verification of the simulation as aforementioned.

Further, the method for evaluating heat treatment using a simulation model sample according to the present invention includes: a step of producing a simulation model sample including a flexible and deformable porous water absorbing material, a small container containing a substance whose properties vary due to heating, the small container being embedded in the porous water absorbing material having water absorbed therein, and a container in which the porous water absorbing material is contained; a step of subjecting the simulation model sample to heat treatment; and a step of measuring a variation of properties of the substance before and after the heat treatment.

According to the aforementioned method for evaluating heat treatment, the small container containing a substance whose properties vary due to heating can be embedded in an arbitrary part of the flexible porous water absorbing material that absorbs water, and thereby enables individual evaluations of the local temperature histories of the porous water absorbing material when the porous water absorbing material is heated.

As one form of the present invention, a sponge may be employed as the porous water absorbing material. Since various types of sponges having different water absorbing properties are easily available, and a sponge having a void ratio and an amount of water absorption according to the type thereof can be used, the sponge is advantageous in that it can easily reproduce the physical properties similar to the actual product.

As one form of the present invention, the simulation model sample may include a substance whose properties vary due to the effect of heat. When the porous water absorbing material having the substance absorbed therein is subjected to heat treatment, and measurement of a variation in the amount or ratio of the substance before and after the heat treatment enable a specific evaluation of the temperature inside the simulation model sample.

### REFERENCE SIGNS LIST

1: Simulation model sample
2: Sponge (flexible porous water absorbing material)
3: Container
4: Insertion port
40: Insertion valve

1. A method for evaluating heat treatment using a simulation model sample, comprising:
a step of allowing a flexible and deformable porous water absorbing material to absorb water and a substance whose properties vary due to water and heating, and the porous water absorbing material to be contained in a container, to produce a simulation model sample;
a step of subjecting the simulation model sample to heat treatment; and
a step of measuring a variation of properties of the substance before and after the heat treatment.

2. The method for evaluating heat treatment using the simulation model sample according to claim 1, wherein the porous water absorbing material is a sponge.
